(19) United States
(12) Patent Application Publication
(10) Pub. No.: US 2012/0204341 A1
(43) Pub. Date: Aug. 16, 2012

(54) Pedicure chair with built-in sterilizing machine placement

(76) Inventor: Quoc Nguyen, Wall, NJ (US)

(21) Appl. No.: 12/931,981

(22) Filed: Feb. 16, 2011

(51) Int. Cl. A61H 35/00; A47C 7/62

(52) U.S. Cl. 4/621; 297/188.11

(57) ABSTRACT

A sterilizing machine of any appropriate type enclosed within a pull-out drawer positioned in a pedicure salon chair forward of its water basin and below its footrest in the occupation of dead space characterizing prior art pedicure chair base designs.

### CROSS-REFERENCE TO RELATED APPLICATIONS

None.

### STATEMENT REGARDING FEDERALLY SPONSORED RESEARCH OR DEVELOPMENT

Research and development of this invention and application have not been federally sponsored, and no rights are given under any Federal program.

### REFERENCE TO A MICROFICHE APPENDIX NOT APPLICABLE

### BACKGROUND OF THE INVENTION

1. Field of the Invention

This invention relates to pedicure salon chairs, in general, and to a pedicure chair having a footbath basin to facilitate pedicure services, in particular.

2. Description of the Related Art

As has been reported, nail salon operations may give rise to un-intended health hazards. If, for example, a salon does not sterilize or disinfect its equipment at all after use, one could be at risk after a manicure or pedicure since the nail salon technician is pushing back on the cuticles in cutting off dead skin; without sterilizing or disinfecting, a salon client may be exposed to any unhealthy condition of a previous client. On the other hand, if the nail salon uses a “UV sterilizer” using ultra-violet rays, such treatments do not work effectively in killing bacteria. As such, they are not generally approved by the Federal Drug Administration for actual sterilization, though they continue to be used.

As will be appreciated by those skilled in the art, however, there are many different steam sterilizers that the nail salon operator can chose from—ranging widely in price as well as in quality. Recognizing that the shorter the cycle of operation the better, some steam sterilizers have very fast cycles, but may cost more than most salons want to spend. A trade-off thus presents itself between quality operation and affordability with these types of sterilizers. For that reason, dry heat machines have been promoted, claimed to be able to sterilize all instruments placed inside its chambers for at least 60 minutes at 320° F. in order to obtain FDA approval.

While all this is readily understandable, it is still not unusual to find sterilization not being offered a client even when such is available. Besides the “affordability” question and the cost involved, observation has revealed an alarming tendency for the nail technician just not getting up from the salon station where treatment is accorded, and walking over to the location where the sterilizer machine sits.

One attempt to deal with this problem is described in my pending U.S. patent application Ser. No. 12/806,680 filed Aug. 19, 2010, and entitled “NAIL SALON WORKSTATION”. There, a manicure table is described having a technician's side, an opposing client's side and a table top work surface; an exhaust motor vacuum, a hose or pipe conduit running upwardly along the table from the vacuum toward the work surface, and a controllable source of illumination at an end of the conduit adjacent to the work surface are provided, for exhausting harmful dust and noxious chemical odors generated at the workstation area. The table top work surface, additionally, is selected optically transparent along its length to allow a client not only to observe the various tools employed by the technician in performing the manicuring function, but also to visualize such items as sterilizing machines, ultraviolet gel machines, and fingernail soak bowls that may be placed on one or more shelves underlying the table top work surface. In such manner, the sterilizing machine of whatever type selected is already there at the work area itself, without any need for the technician to have to walk across the salon shop to where the sterilizer machine may be kept.

As will be recognized by those skilled in the art, the same general problem exists with respect to the use of a pedicure salon chair—where it is equally important to inquire about how files, clippers, cuticle cutters, buffers and permastone instruments are sterilized, and if they are sterilized between client uses. Noting that this is more than just rubbing instruments down with alcohol or hydrogen peroxide, a need for sterilization does truly exist—especially when considered in light of a recent development in which a woman won a jury award of $3.1 million against a nail salon that gave her herpes through the use of non-sterile instruments. While this might be viewed by some as a remote possibility, most dermatologists accept the possible danger of developing nail fungus and/or bacterial infection from manicures and pedicures.

### OBJECTS OF THE PRESENT INVENTION

It is an object of the present invention, therefore, to provide a new and improved type of pedicure salon chair configured to allow a sterilizer machine placement to be built-right-in for a nail technician to access no matter the utensils employed.

It is an object of the present invention, also, to provide this built-in sterilizer machine placement as an alternative to the increasingly employed practice of requiring customer clients to purchase their own instruments and bringing such utilizations along with them each time their nails are to get done.

It is another object of the present invention to provide this built-in placement feature to the pedicure salon chair regardless of the type of machine to be used, and regardless of the actual configuration of the chair.

It is an object of the present invention, additionally, to provide such a pedicure salon chair whether or not the sterilizing machine is steam or dry-heat operative, with or without UV sterilization, and whether the sterilizing machine resembles a toaster oven, a small dishwasher, or whatever.

### SUMMARY OF THE INVENTION

As will become clear from the following description, the present invention discloses a pedicure salon chair with its own built-in sterilizing machine placement. Recognizing that all such pedicure chairs on the market employ a footrest at its front opposite the seat portion and that almost all such chairs are provided with footbath basins for hot and cold water usage in the pedicure operations, the chair of the present invention follows the realization that beneath the footrest and forward of the footbath basin is a large, unused dead space. The sterilizing machine placement of the invention serves as a modification of those pedicure salon chairs presently available for purchase to allow the sterilizing machine to be housed there in what previously represented wasted area and space. As will be seen, a pull-out draw is there located according to the invention, into which any type of sterilizing might be utilized, be it ultra-violet, steam or dry heat.

### BRIEF DESCRIPTION OF THE DRAWINGS

These and other features of the present invention will be more clearly understood from a consideration of the following description, taken in connection with the accompanying Drawings, in which:

FIG. 1 is an illustration of one type of pedicure salon chair available in the prior art;

FIG. 2 is an illustration of a second prior art pedicure salon chair employing a footbath basin for water treatment of a customer client;

FIG. 3 is an illustration of how a built-in sterilizing machine placement can be combined with either type prior art pedicure chair according to the present invention, while

FIG. 4 is an illustration helpful in an understanding how the sterilizing machine can be withdrawn through a pull-out control; and

FIG. 5 is sectional view helpful in an understanding of the placement of the sterilizing machine into the pedicure salon chair itself.

### DETAILED DESCRIPTION OF THE INVENTION

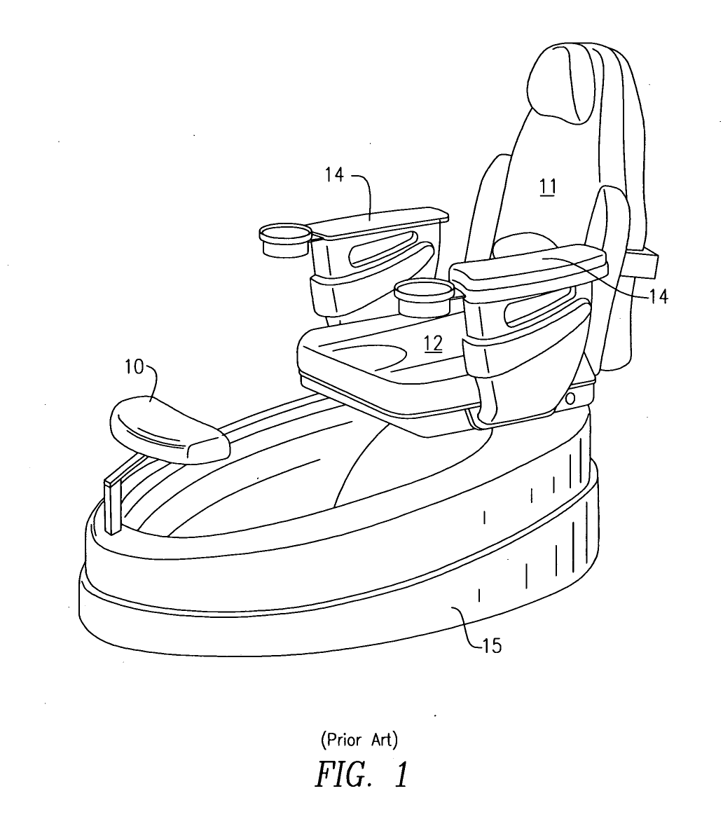

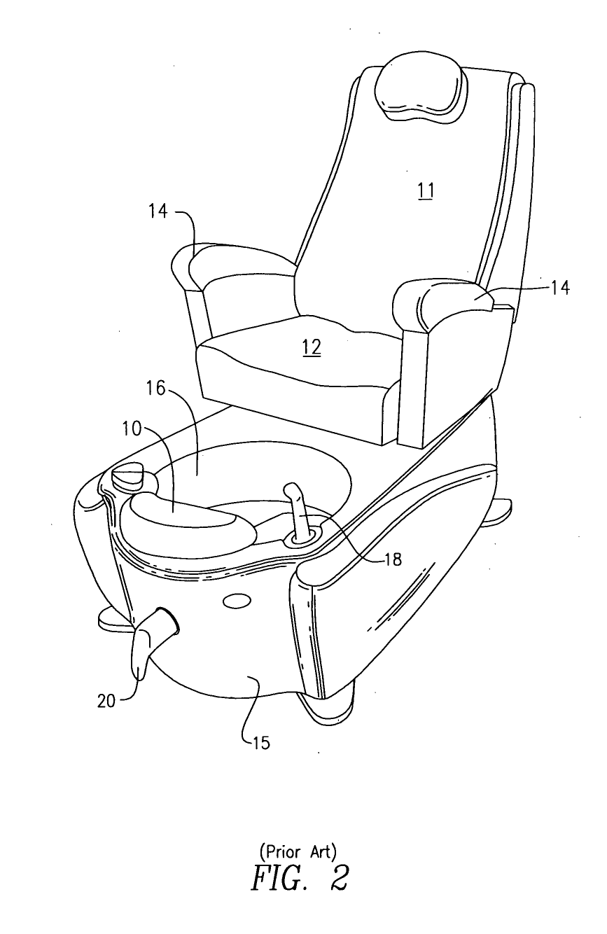

In the prior art pedicure salon chairs of FIGS. 1 and 2, the typically employed footrest is shown by the reference numeral 10 opposite, below and forward of the seat portion 12 where the customer client is positioned, employing armrests 14 and a backrest portion 11. The pedicure chair of FIG. 2 is shown with a base 15 having an included footbath basin 16, to which a water spray may be introduced via a hose unit 18, with a water turn-on input shown at 20—the basin 16 being below the footrest 10 and rearward of it, towards the seat portion 12. Recognizing that the pedicure salon chair of FIG. 1 may be available without such a footbath basin, it is obvious that with the chairs of FIG. 1 and FIG. 2—as well as with others of those characterizing the prior art without footbath basins—a large wasted space exists in the base 15 under their respective footrests, especially with a sideways repositioning of the water input 20 of FIG. 2—which the present invention can put to use.

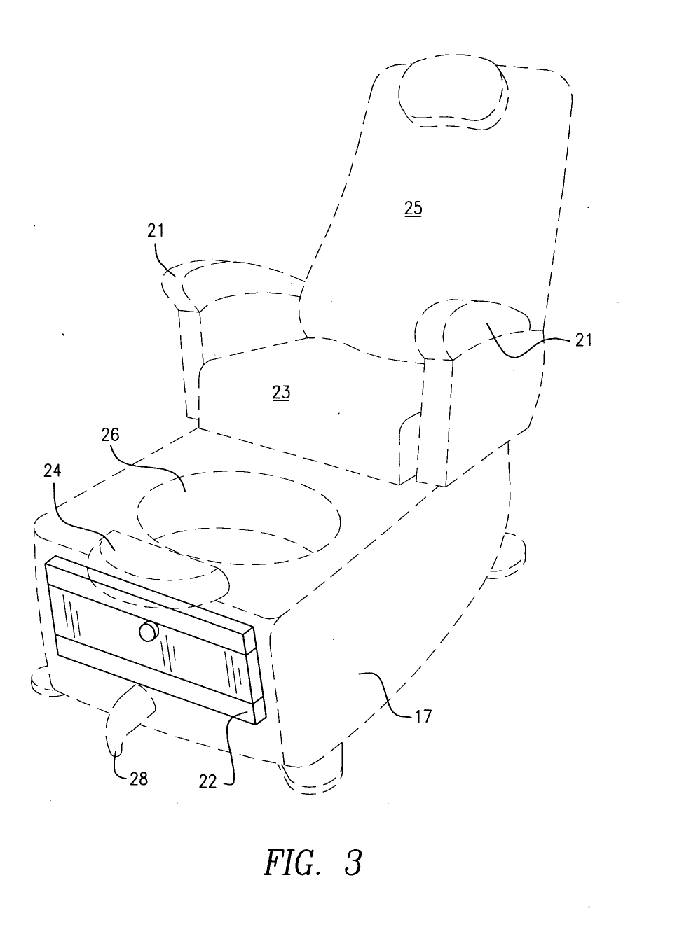

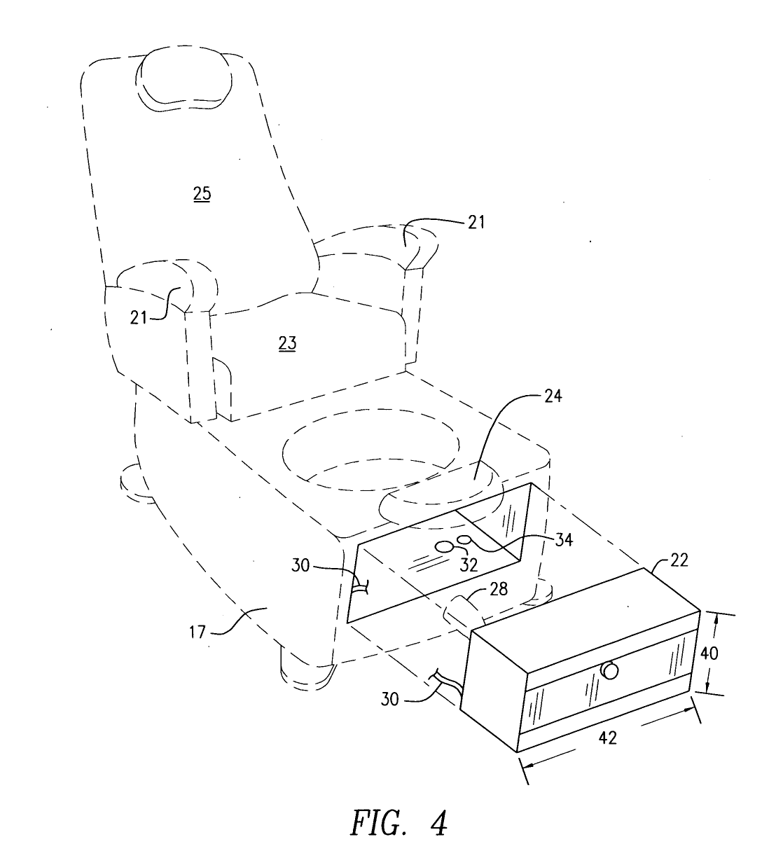

In the FIGS. 3 and 4 illustrations of the invention, the sterilizer machine employed is enclosed within a pull-out drawer 22, available to be slid into and pulled outward from the chair, beneath its footrest 24, in its base 17. Of a depth so as to fall short of its footbath basin 26 when fully closed as in FIG. 3, the drawer 22 would be of a height 40 and width 42 so as to enclose within the base 17 whatever type of sterilizing machine is decided upon to receive the technician's instruments and utensils for sterilization. A water turn-on is shown at 28 in both FIGS. 3 and 4, with reference numeral 30 identifying the electric wiring needed to operate the particular type of sterilizing machine utilized. Reference numerals 32 and 34 in FIG. 4 identify locations at which the water spray may be introduced for use with the footbath basin 26 and an outlet drain for the water removal, respectively. Reference numerals 23 and 25 respectively identify the seat portion of the chair and its back portion, while reference numeral 21 identifies its armrests.

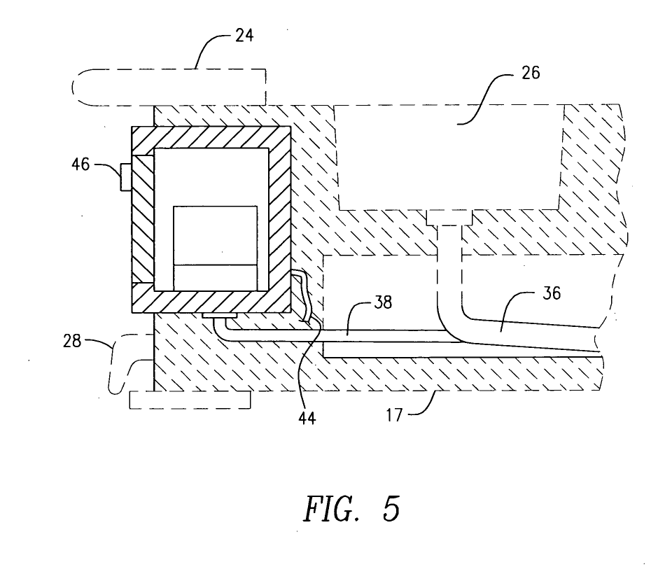

In the sectional-view configuration of FIG. 5, the water outlet from the footbath basin 26 is shown at 36, and an outlet 38 is provided at the base of the sterilizing machine drawer for the situation where a steam type sterilizing machine is employed. In such instance, reference numeral 44 represents an input of water into the sterilizing machine, it being understood that both the hose spray for the footbath basin 26, and the electric wiring for the sterilizing machine have been omitted for the sake of clarity. The drawer pull is shown at 46, and the footrest for the pedicure salon chair is shown at 24. As illustrated, in accordance with the teachings of the invention, the sterilizer machine in its drawer is positioned within the base 17 below the footrest 24 and forward of the footbath basin 26, in previously unused dead space in the pedicure salon chairs which characterize the prior art.

While there has been described what is considered to be a preferred embodiment of the present invention, it will be readily appreciated by those skilled in the art that modifications can be made without departing from the scope of the teachings herein. For at least such reason, therefore, resort should be had to the claims appended hereto for a true understanding of the scope of the invention.

I claim:

1. A salon chair comprising:
a seat portion and a backrest portion;
a base including a footrest located below and forward of said seat portion;
a drawer disposed within said base extending from a first surface thereof a distance towards said seat portion; and
means for pulling said drawer forwardly in an outward direction from said base and for pushing said drawer rearwardly in an inward direction into said base.

2. The salon chair of claim 1 wherein said drawer extends rearwardly from a front surface of said base towards said seat portion a distance falling short of said seat portion.

3. The salon chair of claim 2, including a pedicure instrument/utensil sterilizing machine within said drawer.

4. The salon chair of claim 1 wherein said base also includes a footbath basin located below said footrest, between said footrest and said seat portion, and wherein said drawer extends rearwardly from said front surface of said base towards said footbath basin.

5. The salon chair of claim 4 wherein said drawer extends rearwardly from said front surface of said base towards said footbath basin a distance falling short of said footbath basin.

6. The salon chair of claim 5, also including a pedicure instrument/utensil sterilizing machine within said drawer.

7. The salon chair of claim 6 wherein said base also includes a water inlet source for adding water to said footrest basin.

8. The salon chair of claim 7 wherein said base also includes a water outlet for draining water from said footbath basin.
